(19) United States
(12) Patent Application Publication
(10) Pub. No.: US 2026/0174797 A1
(43) Pub. Date: Jun. 25, 2026

(54) MAGNESIUM AND TRACE ELEMENT OXIDE PREMIX FOR ANIMAL NUTRITIONAL SUPPLEMENTATION, AND MANUFACTURING METHOD

(71) Applicant: TIMAB MAGNESIUM, Dinard (FR)

(72) Inventors: Margaux Tansu, Rennes (FR); Eric Le Fur, Chantepie (FR); Thierry Bataille, Janze (FR)

(73) Assignee: TIMAB MAGNESIUM, Dinard (FR)

(21) Appl. No.: 19/123,282

(22) PCT Filed: Oct. 24, 2023
(86) PCT No.: PCT/FR2023/051668

§ 371 Date: Apr. 22, 2025

(30) Foreign Application Priority Data
Oct. 24, 2022 (FR) FR2211034

(51) Int. Cl. A61K 33/24; A23L 33/16; A61K 31/198; A61K 31/429

(52) U.S. Cl. CPC A61K 33/24; A23L 33/16; A61K 31/198; A61K 31/429; A61K 2300/00; C04B 2235/3217; C04B 2235/3418

(57) ABSTRACT

The invention relates to a synergistic premix for animal nutrition in the form of a ternary solid of magnesium and a trace element, which premix may consist essentially of a solid solution of a magnesium oxide and an oxide of a trace element selected from iron, copper, zinc and manganese. This premix may be obtained by a process comprising a step of preparing a dry mixture of a raw material of the trace element oxide and a raw material of magnesium oxide, then a step of heating the mixture of raw materials to a temperature ranging from 700° C. to 1500° C.

### TECHNICAL FIELD

The invention relates to a nutritional supplement for animals, intended to provide trace elements in addition to the diet. It also relates to a process for the manufacture thereof.

### PRIOR ART

To address diseases and high mortality rates caused by deficiencies and/or toxicities, the agri-food industry offers mineral supplements that are essential for livestock and profitable for the sector.

All living beings have a beginning of digestive tract at pH=6-8 controlled mainly by saliva. Food then passes into the stomach at pH=2-3 except for ruminants, for which food first undergoes bacterial fermentation in the rumen. Upon leaving the stomach, food arrives in the intestine at pH 6-7 where it is bio-assimilated.

The absorption or bio-assimilation of minerals, defined as the fraction of the chemical element passing from food to the portal blood circulation, occurs mainly through the intestinal mucosa, with the exception of magnesium which is absorbed almost entirely at the reticulo-rumen in ruminants.

A trace element deficiency in the body causes a chronic or episodic deficiency leading to more or less serious symptoms. This deficiency is frequently observed in animals, because trace elements are generally poorly absorbed by the body at the duodenum, due to several factors. Indeed, the proteins and fibers in the ration, precipitating agents and mineral competition limit bio-assimilation by the body by complexing the trace elements in ionized form.

Zinc, as well as other trace elements, are poorly assimilated, mainly due to molecular complexations occurring in the stomach of animals. Indeed, trace element salts administered as a supplement to the ration have high dissolution kinetics in an acid environment, so that the trace element solubilized in cationic form complexes with the organic matter present in the stomach and can no longer reach the bio-assimilation zone. The ionized trace element binds in the stomach to chelating agents present in the ration such as phosphates, oxalates, fats and high molecular weight peptides before reaching the duodenum, which leads to a significant drop in the bio-assimilated amounts. Among the chelating agents, phytic acids and their base forms (phytates) are phosphorus compounds naturally present in cereals and legumes in the diet, which bind with minerals such as iron, zinc, copper, cobalt or manganese, to create insoluble complexes when leaving the stomach, so that the trapped minerals are no longer bio-assimilable in the duodenum. Indeed, the pH influences the solubility of the mineral-phytate complexes formed: in the case of zinc, for example, zinc-phytate complexes are insoluble at pH values above 4.3.

A purpose of the present invention is to provide a supplementation tool which improves the bio-assimilation of trace elements, and which allows to reduce the amount of trace element added to the ration without affecting the functional performance of the animal.

The supplements of the prior art are subject to improvement given the limited bio-assimilation of certain trace elements, even in the case of excessive intakes. A large part of these intakes are not used by animals and are released into the environment, thus inducing negative ecological consequences. For example, soil contamination by minerals such as zinc in large poultry, pig and cattle farms has been documented. To reduce environmental pollution related to the intake of zinc in the agri-food industry, it would therefore be desirable to improve the bioavailability of zinc.

There is therefore a need to use more environmentally friendly trace element food supplements the use of which involves limited soil contamination.

Still from an ecological perspective, it would be beneficial if the trace element supplement could be manufactured using a preparation process with a limited impact on the environment.

The strategy of providing excess trace elements in the ration to compensate for their low bioavailability has been adopted by the majority of players in the agri-food sector. However, the risks of overdosage are not without danger for the health of the animal.

The diversity of zinc sources used for animal nutrition is significant. The raw materials used as a source of zinc in animal nutrition are mainly zinc sulfate and zinc oxide. For example, the share of zinc oxide as a source of zinc in poultry farming is estimated at 80%.

Many means have been proposed in the prior art to improve the bioavailability of inorganic zinc sources.

A first avenue consisted of modifying the raw material by proposing nano-Zinc, zinc oxide with a high specific surface or microencapsulated zinc salts.

Another strategy proposed in the prior art consisted in adding bioavailability enhancers to the inorganic zinc source, such as sugars, animal proteins and polysaccharides, which can improve the bioavailability of the minerals. Organic zinc sources used in supplementation in the form of complexes, chelates, proteinates or polysaccharides generally lead to better bioavailabilities than inorganic sources, but their manufacturing cost remains high.

The use of organic zinc sources, such as zinc glycinate, has also been proposed. However, since the zinc content of organic solutions cannot exceed 25% by mass due to the number of atoms present in the organic molecule, premix formulators favor inorganic zinc sources to maintain the possibility of adding supplements thereto and thus be able to offer multi-functional supplements. Furthermore, these products are relatively expensive.

It would therefore be desirable to propose a trace element supplementation that is more profitable from an economic point of view for breeders, and which allows to limit the amount of trace element administered to the animal with equal zootechnical performance.

### DISCLOSURE OF THE INVENTION

The invention meets these needs and relates to a synergistic premix for animal nutrition comprising a ternary solid of magnesium, trace element and oxygen, which may be in the form of a solid solution of a magnesium oxide and a trace element oxide, said trace element being selected from iron, copper, zinc and manganese.

The inventors have surprisingly discovered that the synergistic premix comprising a ternary solid of magnesium, trace element and oxygen, which may be in the form of a solid solution of a trace element oxide and a magnesium oxide improves the penetration of the trace element through the intestinal cells in vitro, and demonstrates better availability compared to the trace element oxide alone, at equivalent dose.

The synergy of action between the two oxides allows to delay the solubilization kinetics of the trace element, and to limit the complexation of the latter by the organic matter of the ration at the digestion stage in the stomach, for example its complexation with phytates. The premix of the invention therefore allows to maximize the probability of absorption of the trace element at the duodenum by synergy with magnesium, which acts as a support and a means for physical protection of the trace element in the digestive tract.

The premix of the invention comprising a trace element has the advantage of being manufactured by a process carried out using the dry route, the environmental impact of which is reduced compared to solvent-phase processes.

In a particular embodiment, the invention provides a food supplementation tool which improves the bio-assimilation of trace elements such as zinc in animals, in particular livestock such as ruminants, poultry and pigs.

The premix of the invention can be very advantageously administered in an amount such that the daily intake of trace element in the ration is lower than the doses of trace element oxide practiced in the prior art, without affecting the functional performances of the animal, in particular the performances of growth, weight gain, feed efficiency, ration conversion index and nutrient assimilation.

The premix of the invention has the further advantage of reducing the amount of zinc released into the environment, and of helping to limit pollution of soil, water and the food chain by heavy metals.

The inventors have surprisingly discovered that the synergistic premix of the invention delays the solubilization of trace element salts, and can consequently overcome the unfavorable complexations that occur at acid pH between the solubilized trace elements and certain molecules of the food ration. The premix of the invention with adequate solubilization kinetics allows to increase the proportion of trace elements in ionic form at the end of the passage through the stomach, just before arrival in the intestine, and thus allows to increase the bioavailability of the trace elements.

In the premix of the invention, the trace element oxide has suitable solubility kinetics, solubilization being delayed in the stomach but sufficiently rapid to make the trace element available in ionic form at the exit of the stomach, before entering the place of absorption, the intestine.

The inventors surprisingly discovered that using a magnesium-based compound can increase the absorption of zinc.

### BRIEF DESCRIPTION OF THE DRAWINGS

FIG. 1 shows the amounts of zinc measured in the feces of piglets supplemented either with a ZnO—MgO mixture according to the invention or with a ZnO of the prior art.

FIG. 2 shows the curve of the evolution of the pH value of a hydrochloric acid solution comprising a ZnO—MgO mixture according to the invention or a comparative mixture.

### DESCRIPTION OF THE EMBODIMENTS

The invention relates to a synergistic premix for animal nutrition, said premix comprising magnesium oxide and a trace element. The premix may be a ternary solid comprising magnesium atoms and trace element atoms.

For the purposes of the invention, the term trace element means a chemical element in ionic or atomic form selected from zinc, copper, cobalt, manganese, iron, iodine and selenium. Magnesium is a macroelement, not a trace element.

The term “ternary solid” means a crystalline mineral solid comprising at least one crystalline phase in which magnesium atoms, trace element atoms and atoms of a third element are distributed. In a particular embodiment, the ternary solid is a ternary oxide of magnesium and at least one trace element selected from iron, copper, zinc and manganese. The term “at least one” means one or more.

The molar ratio between magnesium and the trace element in the ternary solid is preferably greater than 1 and greater than 0.1.

The ternary solid preferably consists essentially of atoms of magnesium, oxygen, a trace element and optionally phosphorus, hydrogen, carbon and sulfur. The ternary solid may have the chemical formula (MgₓX_{y}Y_{z}), Y representing O, CO₃, OH, SO₄ H₂PO₄, or PO₄, X representing a trace element atom selected from iron, copper, zinc and manganese, and x, y and z being decimal numbers. The ternary solid may thus be a ternary oxide when Y represents O, or a ternary phosphate when Y represents P₃O₄.

The word “essentially”, in particular the word “essentially consisting” in the present description, regardless of the characteristic to which it refers, an amount greater than a value selected from 90%, 95%, 98% or 99%, the percentage being able to be expressed in mass, in moles or in volume.

The crystalline form of the ternary oxide may be the same as or different from the crystalline form of a chemical compound of magnesium comprising oxygen atoms, such a compound being referred to as “a magnesium oxide” in the remainder of this description, magnesium oxide (MgO) being a particular magnesium oxide.

In the present description, the term “magnesium oxide” means a material comprising magnesium and oxygen atoms. A magnesium oxide may comprise a magnesium hydroxide, a magnesium carbonate, magnesium oxide, a magnesium carbonate, or a mixture thereof. For example, a magnesium oxide within the meaning of the invention comprises magnesium oxide (MgO), magnesium carbonate (MgCO₃), a magnesium phosphate (comprising Mg, P, O and optionally H), magnesium hydroxide (Mg(OH)₂), and their hydrates.

In a particular embodiment, the crystalline form of the ternary oxide may be different from the crystalline form of magnesium oxide (MgO), such as the crystalline form of periclase. The crystalline form of the ternary oxide may be different from the crystalline form of a trace element oxide. Finally, the ternary oxide preferably consists essentially of magnesium, oxygen and said trace element.

When the trace element is zinc for example, the ternary oxide may have a crystalline form different from that of a magnesium oxide, and different from that of a zinc oxide (that is to say a compound comprising zinc atoms and oxygen atoms), the zinc oxide being for example selected from ZnO (zinc oxide), ZnCO₃ (zinc carbonate), zinc hydroxycarbonate, and ZnSO₄ (zinc sulfate).

According to a particular embodiment of the invention, the ternary solid is a solid solution, more particularly a substitution solid solution.

“Solid solution” means: a homogeneous solid in which a magnesium oxide, such as magnesium oxide, and a trace element oxide, such as trace element oxide, form a single crystalline phase. The observation of the crystalline structure and the homogeneity of the solid solution and/or those of the premix may be carried out under a microscope according to a method known to the person skilled in the art.

The solid solution may be a substitution solid solution of the trace element in a magnesium oxide, such as magnesium oxide. A “substitution solid solution” means a solid solution in which atoms of the trace element take the place of magnesium atoms in the crystal lattice of the solid solution.

Thus, the synergistic premix for animal nutrition of the invention comprises a solid solution essentially consisting of a magnesium oxide and a trace element oxide, said trace element being selected from iron, copper, zinc and manganese. The solid solution may be a solid solution of substitution of the trace element in a crystalline magnesium oxide, a magnesium oxide being selected from magnesium oxide (MgO), magnesium carbonate (MgCO₃), a magnesium phosphate (comprising Mg, P, O and optionally H), or magnesium hydroxide (Mg(OH)₂). In the premix of the invention, the magnesium may advantageously provide physical protection of the trace element, such as zinc for example. The magnesium advantageously serves as a support, a biological vector or a retarder of dissolution of the trace element in the digestive tract of an animal.

The molar amount of the trace element in the premix can vary.

For example, when the trace element is zinc, zinc may represent from 1% to 45% by mole of the premix. When the trace element is copper, copper may represent from 1% to 25% by mole of the premix.

The amount of the trace element in the ternary solid, expressed in moles or in mass, is advantageously greater than a limit value selected from the group consisting of 1%, 2%, 3%, 4%, 5%, 6%, 7%, 8%, 9% or 10%.

When the trace element is zinc, the zinc may represent from 1% to 45% by mole, preferably from 3% to 45% by mole, or even from 10% to 45% by mole of the ternary solid. Zinc may represent from 10% to 45% by mole of a solid solution consisting of magnesium, oxygen and zinc.

When the trace element is copper, the copper may represent from 1% to 40% by mole, preferably from 3% to 25% by mole, or even from 10% to 25% by mole of the ternary solid of the ternary solid. In a particular case, the copper may represent from 10% to 25% by mole of a solid solution consisting of magnesium, oxygen and copper.

When the trace element is iron, the iron may represent from 1% to 45% by mole, preferably from 3% to 40% by mole, or even from 10% to 30% by mole of the ternary solid. The iron may represent from 10% to 30% by mole of a solid solution consisting of magnesium, oxygen and iron.

When the trace element is manganese, the manganese may represent from 1% to 45% by mole, preferably from 3% to 40% by mole, or even from 10% to 30% by mole of the ternary solid. The manganese may represent from 10% to 30% by mole of a solid solution consisting of magnesium, oxygen and manganese.

The invention also relates to a process for manufacturing a synergistic premix for animal nutrition, said process comprising a step of heating a mixture comprising a powdery raw material of a magnesium oxide, and a powdery raw material of a trace element, in particular a powdery trace element oxide raw material. The heating step may also be referred to as sintering or heat treatment in the present description.

The magnesium oxide raw material is of food grade. The magnesium oxide raw material is of food grade in that it meets the requirements of European Regulation 767/2009 concerning the placing on the market and use of animal feed, European Directive 2002/32/EC and Regulations (EU) No 574/2011 and (EU) No 277/2012 concerning the levels of undesirable substances for animal nutrition. In particular, the amount of magnesium oxide in the raw material is greater than or equal to 70%, for example comprised between 70% and 99%, and the content of heavy metals is less than 500 ppm, more preferably less than 350 ppm.

In a particular embodiment, the magnesium oxide raw material comprises a content, expressed by mass or by mole, of less than 10%, preferably less than 5%, more preferably less than 1%, of a trace element selected from iron, copper, zinc and manganese.

As raw material, it is possible to use a magnesium oxide, a magnesia MgO, a magnesium carbonate, a magnesium hydroxycarbonate such as hydromagnesite (Mg₅(CO₃)₄(OH)_{2,4} H₂O), a magnesium sulfate, a magnesium phosphate, a magnesium chloride, a magnesium hydroxide or one of their hydrates.

In one embodiment of the invention, the particle size value of the magnesium oxide raw material and the particle size value of the trace element oxide raw material are preferably less than 200 microns.

“Particle size value” means a value selected from the maximum value of the particle size distribution, the value of D50 of the particle size distribution (50% of the particles of the raw material having a diameter less than the value D50), the value of D90 of the particle size distribution (90% of the particles of the raw material having a diameter less than the value D90), the value of D99 of the particle size distribution (99% of the particles of the raw material having a diameter less than the value D99), and the value of the mesh size of the raw material. The particle size value can be measured by any method known to the person skilled in the art.

The value of the particle size of the magnesium oxide raw material is preferably less than 200 microns, for example less than 100 microns, or even less than 50 microns or 25 microns.

In a particular embodiment, the D50 of the magnesium oxide raw material is less than 100 microns.

The D90 and/or D99 of the magnesium oxide raw material can be less than 200 microns.

Examples of magnesium oxide raw materials are the product MgO marketed by Sigma-Aldrich (Code: 342793 and CAS: 1309-48-4) with a purity greater than 99% and a mesh size of approximately 44 micrometers (equivalent to 235 mesh), and magnesium hydroxycarbonate (with a reference Honeywell 63062, CAS 39409-82-0) with an equivalent purity in MgO≥70%, containing less than 50 ppm of zinc and a density of 2.16 g/cm³ (20° C.).

The magnesium oxide raw material can also be selected from the product Timag Reactive® FT2013, the product Timag MAG® PEV, the product Brazamag® HR FT 2421 and the product Brazamag® FT 2402, all marketed by the company Timab Magnesium.

In one embodiment, the magnesium oxide raw material is magnesia.

The value of the particle size of the magnesia is for example such that the D50 is less than a value selected from 100 microns, 50 microns and 25 microns. In this embodiment, the D90 or the D99 of the magnesia may further be less than 200 microns.

The magnesia is preferably a caustic magnesia obtained by calcining magnesium carbonate at a temperature comprised between 900° C. and 1300° C. The caustic magnesia may comprise, in addition to MgO, impurities such as CaO, SiO₂, Fe₂O₃ and Al₂O₃.

The trace element oxide raw material is preferably of food grade, the definition of the term “food grade” being in accordance with the definition given above.

The trace element oxide raw material is preferably characterized by a minimum value of the mass percentage of trace element selected from 60%, 70%, 80% or even 90%, the mass percentage being expressed relative to the mass of the raw material.

In one embodiment of the invention, the particle size value of the trace element oxide raw material is less than 200 microns, for example less than 100 microns, or even less than 50 microns or 25 microns. The definition of the term “particle size value” in the context of describing the trace element oxide raw material is consistent with the definition of the term given above in the context of describing the magnesium oxide raw material.

The trace element oxide raw material preferably has an impurity content, expressed by mass or mole, of less than 10%, preferably less than 5%, relative to the amount of trace element raw material.

The trace element oxide raw material has, for example, at least one of the following physicochemical characteristics: a particle size value of less than 200 microns, a trace element content of more than 60% by mass, or an impurity content of less than 10% by mass.

The trace element oxide raw material may predominantly comprise a trace element oxide. The term “trace element oxide” means a material comprising atoms of the trace element, oxygen, and optionally sulfur, carbon and hydrogen. The trace element oxide raw material may be a raw material comprising a trace element hydroxide, a trace element carbonate, a trace element oxide, a trace element carbonate, or a mixture thereof.

In the case of zinc for example, the trace element oxide can thus be selected from ZnO (zinc oxide), ZnCO₃ (zinc carbonate), zinc hydroxycarbonate such as for example hydrozincite Zn₅(CO₃)₂(OH)₆, and ZnSO₄. In a particular embodiment, the trace element oxide is ZnO.

Examples of zinc oxide raw material are the product ZnO supplied by the company Acros Organics under the reference Zinc oxide, ACS Reagent® (CAS: 1314-13-2), zinc hydroxycarbonate sold by Sigma Aldrich under the reference Zinc Carbonate Basic® with the formula [ZnCO₃]₂·[Zn(OH)₂]₃ (CAS: 5263-05-5) or granulated zinc oxide of brand ZnO-KB® sold by the supplier Silar.

The amount of zinc oxide raw material is preferably selected so that the zinc element represents, for example, 1% to 40% by mole of the sum of the number of moles of the zinc element of the zinc oxide raw material, and the number of moles of the magnesium element of the magnesium oxide raw material.

In the case of iron, the trace element oxide may be selected from FeCO₃, FeO, Fe₂O₃, Fe₃O₄, FeHO₂, Fe₄H₆O₉. Iron carbonate may be anhydrous or hydrated. An iron oxide raw material may be Fe₂O₃ of purity greater than 95% and particle size value less than 5 microns.

When the trace element is manganese, the trace element oxide may be selected from MnCO₃, MnO, Mn₂O₃, MnO₂, Mn₃O₄, Mn(OH)₂. Manganese carbonate may be anhydrous or hydrated.

Finally, a copper oxide can be selected from CuCO₃, CuO, Cu₂O and Cu(OH)₂, the copper carbonate being able to be anhydrous or hydrated.

The trace element raw material, used in particular as a source of trace element oxide, may be any natural mineral raw material known to the person skilled in the art comprising the trace element or a trace element oxide. The raw material will have undergone, after extraction, physical treatments such as grinding, screening, granulation, leaching, purification, chemical treatment, drying, calcination and sieving.

In one embodiment of the invention, the particle size of the trace element oxide raw material, and the particle size of the magnesium oxide raw material, both defined as D90, are less than 200 microns.

The heating step of the process of the invention may comprise a temperature increase from room temperature to a plateau temperature ranging from 700° C. to 1500° C., a maintenance of the plateau temperature for a period ranging from 1 hour to 600 hours, and a temperature decrease from the plateau temperature to room temperature. The maximum temperature reached during sintering is preferably comprised between 1100° C. and 1500° C., in particular in the case of a zinc oxide raw material.

The rate of temperature rise and/or fall is in particular comprised between 5° C./min and 30° C./min, for example between 10° C./min and 20° C./min. The total duration of the heat treatment is advantageously comprised between 1 h and 600 h, preferably between 3 h and 48 h.

In addition to the sintering step, the process of the invention may comprise a subsequent step consisting of an annealing step. This annealing step may be followed by an overprotection step with magnesium hydroxycarbonate (hydromagnesite).

The present application also relates to a premix capable of being obtained by the manufacturing process described above.

A synergistic premix for animal nutrition may be obtainable by a process comprising a step of preparing a mixture consisting of dry mixing, preferably at room temperature, a raw material of trace element oxide and a raw material of magnesium oxide, then calcining the mixture.

The premix of the invention may be obtainable by a process comprising a step of preparing a mixture consisting of dry mixing a raw material of powdered food grade trace element oxide, and a raw material of powdered food grade magnesium oxide, then a step of heating said mixture to a temperature ranging from 700° C. to 1500° C. in a closed container, in the absence of water, for a period ranging from 1 to 600 hours, for example for a period ranging from 3 to 48 hours.

The premix of the invention may be in various forms including a lick bucket, a lick block, a powder, a suspension or a solution. It may be introduced directly into the animal's ration or drink.

Alternatively, it may be formulated into a food or dietary supplement that comprises other ingredients.

The invention thus relates to a nutritional supplement for non-human animals comprising the premix described above, and a compound selected from vitamins, probiotics, macroelement salts, trace element salts, enzymes and amino acids.

The invention will also relate to a method for preparing a food ration for a non-human animal, comprising a step of dry incorporation of the premix according to claim 1 with fodder and/or cereals.

The premix of the invention is intended for feeding livestock or breeding animals, in particular ruminants, poultry, pigs, and any other animal species for which zinc supplementation is desirable.

The premix doses of the invention may be different depending on the species, and depending on the type of production in the case of breeding, for example milk and/or meat. The doses may also vary depending on the stage of production at which the breeding is. The person skilled in the art will know how to adapt the necessary doses depending on the intended use.

For example, ruminants are fed mainly on forage and a little cereal, but since the magnesium intake from forage often remains too low, systematic mineral supplementation is recommended for these animals, the recommended daily intake being of the order of 100 mg/kg to 10 g/kg of dry matter (or mg/kg DM) of the ration, for example 300 mg/kg DM to 500 mg/kg DM (or ppm DM).

In species that consume more cereals, such as pigs and poultry, magnesium can be used occasionally, as needed, with a recommended daily intake of 600 ppm and 1000 ppm DM respectively.

Zinc supplementation may be recommended in female ruminants to ensure reproductive performance, but also in poultry and pigs. The recommended amounts of zinc are similar for these three species and are between 30 ppm and 150 ppm.

In accordance with Regulation EC 1095/2016, the intake of zinc in feed may be limited to 150 mg Zn/kg of complete feed for piglets, sows, rabbits and fish, to 200 mg Zn/kg of complete feed for cats and dogs, and to 120 mg Zn/kg for other species.

The premix of the invention allows to obtain at least one biological effect in a non-human animal, this effect being able to be a beneficial effect on the animal's breeding performance. Indeed, a synergy of action is observed between magnesium oxide and trace element oxide.

The biological, non-therapeutic effect may be selected from increasing the weight gain of the animal, supporting the growth of the animal, improving feed efficiency, improving the feed conversion index and supporting the assimilation of nutrients. The invention relates for example to the use of the premix described above for increasing the weight gain of an animal, preferably at the beginning of weaning.

The non-human animal is in particular selected from livestock, racing animals, and domestic animals, such as for example poultry, crustaceans, fish, dogs, cats, horses, rabbits, sheep, goats, ruminants and pigs.

One embodiment of the invention generally relates to the use of an amount of premix to obtain a significant increase of at least 5%, or even at least 10%, in a biological performance effect in an animal, compared to the biological effect obtained with an equal amount of trace element oxide raw material.

The “amounts” within the meaning of the invention which relate to the uses of the premix are molar amounts expressed in moles of trace element or mass amounts, the mass amounts being able to be those of the premix or those of a trace element oxide.

Another embodiment of the invention relates to the use of an amount of premix of the invention for obtaining a biological performance effect in an animal and/or a value of this effect, which is identical to that obtained with an equal amount of trace element oxide raw material.

The present description also proposes the use of the premix described above to reduce the amounts of trace elements which are released by animals into the environment via feces, and/or to limit the pollution of livestock soils by trace elements, which may be heavy metals.

The premix of the invention can be used to obtain at least one biological effect that is significantly improved in comparison with the same biological effect of a trace element source of the prior art, at identical doses of trace element in the premix of the invention and dose of trace element in the trace element source, in the sense that the molar number of trace element in the dose of premix of the invention and the molar number of trace element in the dose of trace element source are identical. The trace element source of the prior art is for example selected from inorganic salts of trace elements and organic salts of trace elements.

The biological effect can be selected from the penetration of the trace element through the intestinal cells; limitation of complexation of the trace element with organic matter contained in the digestive tract, in particular, limitation of complexation of the trace element with phytates; increase in bioavailability of the trace element; delayed release of the trace element into the body; delay in the kinetics of solubilization of the trace element oxide in the stomach; and increase in absorption of the trace element at the duodenum.

The ternary solid allows in particular a delayed release of the trace element. In comparison, a trace element which is not in the form of a ternary solid does not cause a delay in dissolution and undergoes a very rapid, almost instantaneous solubilization in an acid medium.

For example, a ternary solid that is capable of being obtained by heating at a temperature ranging from 700° C. to 1500° C. a powder mixture of a magnesium oxide raw material and a trace element oxide raw material advantageously has a kinetics of release of the trace element in bio-assimilable form that is delayed in comparison with a reference product, which corresponds to the powder mixture used to prepare the ternary solid. According to a particular embodiment, the ternary solid allows to delay the release of the trace element in an acid medium, typically in an acid solution whose initial pH is comprised between 1.0 and 2.0.

For the purposes of the invention, the term “delayed release” means a start of release of the trace element in bio-assimilable form contained in the ternary solid which is later than that of the trace element which is contained in the reference product. The term “delayed release” may also correspond to a duration at the end of which at least 90% by mole of the trace element contained in the ternary solid are released, said duration being greater than the duration at the end of which at least 90% by mole of the trace element contained in the reference product are released.

According to a particular embodiment, the delayed release kinetics is evaluated in vitro by dissolving the ternary solid in an acid solution whose initial pH is comprised between 1.0 and 2.0. The dissolution of the trace element in the acid solution can occur in a window ranging from 30 s to 300 s, for example from 50 s to 250 s, from the introduction of the ternary solid into the acid solution, while the reference product mentioned above begins to dissolve as soon as the ternary solid is introduced into the acid solution. The time after which the value of the amount of trace element dissolved from the solid solution reaches the value of the amount of trace element dissolved from the product can be advantageously increased by at least 50 s, preferably by at least 100 s, and more preferably by at least 150 S.

In particular, the premix of the invention containing a ternary oxide of zinc and magnesium allows to obtain an effect superior to that of zinc oxide alone, with equal amounts of zinc in the premix and in the zinc oxide, administered to an animal.

The premix of the invention containing zinc as a trace element may have particularly advantageous solubilization kinetics, defined according to the following conditions, TO being the time of introduction of the premix of the invention into a hydrochloric acid solution at pH=2.5:
total insolubilization of Zn²⁺ (zero molar concentration of Zn²⁺) between T0 and T100 minutes, and total solubilization of Zn²⁺ at T 120 minutes,
solubilization of Zn²⁺ less than 50 mol %, preferably less than 40 mol %, at T 30 minutes and total solubilization at T120 minutes.

The present application describes a synergistic premix for animal nutrition, said premix comprising a solid solution essentially consisting of a magnesium oxide and a trace element oxide, said trace element being selected from iron, copper, zinc and manganese, the premix being capable of being obtained by the process comprising a step of preparing a mixture consisting of dry mixing a powdered food grade trace element oxide raw material, and a powdered food grade magnesium oxide raw material, then a step of heating said mixture to a temperature ranging from 700° C. to 1500° C. in a closed container, in the absence of water, for a period of 3 to 48 hours. The particle size of the trace element oxide raw material, and the particle size of the magnesium oxide raw material, both defined as D90, are advantageously less than 200 microns. The solid solution may be a solid solution for substituting the trace element in magnesium oxide. When the trace element is zinc, and the zinc may represent from 1% to 45% by mole of the premix. When the trace element is copper, and the copper may represent from 1% to 25% by mole of the premix.

The invention will be described in more detail in the following examples. Unless otherwise stated, the temperature is comprised between 20° C. and 25° C., and the pressure is equal to 1 bar.

### Example 1: Preparation of a Premix According to the Invention Comprising Zinc: ZnO—MgO

47.6% by mass of [ZnCO₃]₂·[Zn(OH)₂]₃ (CAS: 5263-05-5) supplied by Sigma-Aldrich and 52.4% by mass of MgO (CAS: 1309-48-4) of 99% purity also supplied by Sigma-Aldrich are dry mixed.

The mixture was heated from room temperature to a temperature of 1000° C. in a container at a rate of 20° C./min, then maintained at this temperature for 16 hours. The amount of Zn in the obtained premix was 25 mol %.

### Example 2: Preparation of a Premix According to the Invention Comprising Iron: Fe₂O₃—MgO

45.8% by mass of Fe₂O₃ (CAS: 1345-25-1) with a purity >96% and a particle size <5 μm supplied by Sigma-Aldrich, and 54.2% by mass of MgO (CAS: 1309-48-4) with a purity of 99% also supplied by Sigma-Aldrich are dry mixed.

The mixture was brought from room temperature to a temperature equal to 950° C. in a closed container at a rate of 12° C./min, then maintained at this temperature for 4 hours. The amount of Fe in the obtained premix was 30 mol %.

### Example 3: Preparation of a Premix According to the Invention Comprising Copper: CuO—MgO

25.8% by mass of Cu(II) O (CAS: 1317-38-0) from batch 329902/1 supplied by the company Fluka Chemika (purity greater than 98%) and 74.2% by mass of MgO (CAS: 1309-48-4) of 99% purity also supplied by Sigma-Aldrich were dry mixed.

The mixture was brought from room temperature to a temperature equal to 870° C. in a closed container at a rate of 7° C./min, then maintained at this temperature for 4 hours. The amount of Cu in the obtained premix was 15 mol %.

### Example 4: Preparation of a Premix According to the Invention Comprising Manganese: MnO—MgO

43% by mass Mn(II) O (CAS: 1344-43-0) with a purity greater than 99% supplied by Sigma-Aldrich and 57% by mass MgO (CAS: 1309-48-4) with a purity of 99% also supplied by Sigma-Aldrich were dry mixed.

The mixture was brought from room temperature to a temperature equal to 950° C. in a closed container at a rate of 20° C./min, then maintained at this temperature for 6 hours. The amount of Mn in the obtained premix was 30 mol %.

### Example 5: Test of In Vitro Digestion of the Premix According to the Invention ZnO—MgO, and Comparison with the Prior Art

The objective of this test was to determine the solubility of Zinc in feed supplemented with different zinc salts, including the ZnO—MgO premix of the invention. More specifically, the solubilization of zinc in an in vitro digestion model was compared, using the premix of Example 1, standard zinc oxide ZnO with commercial reference ZnO 72% from the company Arkop, zinc oxide with commercial reference HiZox® from the company Animine, zinc sulfate with a purity greater than 99%, or zinc glycinate of brand E.C.O Trace® manufactured by the company Biochem.

### Dissolution Protocol:

This in vitro model allows to reproduce the chemical conditions that apply to a food traveling through the digestive tract of an animal. Indeed, the pH, the retention time, the nature and the intensity of the constraints are important parameters to take into account to evaluate the bioavailability of zinc.

The parameters selected for the Mg²⁺ and Zn²⁺ solubilization protocol were established by comparison with the data found in the literature relating to the stomach of ruminants. Thus, the volume of the abomasum is approximately ten liters and our volume considered in the reactor is 200 milliliters. The mass of product introduced allows to achieve the equivalent concentration of 5000 ppm of the element in the reaction medium. Since the pH of the abomasum is generally comprised between 2 and 3, the pH of the reaction medium in the laboratory is set at 2.5 by adding hydrochloric acid at a flow rate of up to 10 mL/min to simulate the supply of gastric juice into the stomach. The reaction medium is maintained at 39-40° C. corresponding to the internal temperature of the species considered. More precisely, a 200 mL solution of hydrochloric acid at pH=2.5 is prepared in a 1 liter beaker. This beaker is placed in a water bath heated to 40° C., itself placed on a stirring plate. An automatic titrator is programmed to bring the pH back to 2.5 with a maximum flow rate of 10 mL/min with a pH control range equal to 0.5. The titrator is filled with a 0.1 mol/L hydrochloric acid solution. The 20 mL dosing pump is rinsed 3 times with the 0.1 mol/L hydrochloric acid solution. The pH-metric probe of the titrator is then calibrated with a pH=7 buffer and a pH=4 buffer. The electrode and the injector are then placed in the solution to be analyzed, taking care to contact the injector with the solution to reduce response times. 1 gram of the solid sample is then introduced into the 200 mL HCl solution at pH=2.5. Stirring is started at the same time as a stopwatch and the titrator. Stirring is carried out using a four-centimeter magnetic bar in order to stir as much of the beaker surface as possible. The experiment is carried out over two hours by taking 2 mL of solution at different kinetic times. The volumes taken with the syringe are then filtered through 0.45 μm filters then 125 UL of the filtered solution is introduced into a 200 ml flask. Finally, 10% of the volume of the flask is made up with the strontium solution then the flask is topped up to the mark with distilled water.

### Sample Preparation:

The feed consisted of a typical pig mix: 33% wheat, 32% corn and 35% soybean meal. The feed was first ground then mixed with each zinc oxide source in a Lodige® mixer for 60 seconds.

Each zinc salt was supplemented at 1640 ppm in the feed.

An assay was performed by Inductively Coupled Plasma Mass Spectrometry (ICP-MS), to ensure the homogeneity of the mixture.

### In-Vitro Digestion Protocol

The supplemented food was subjected to in-vitro digestion comprising two steps: in a pre-stomach solution at pH=7.0, then in a gastric solution at pH=2, and finally in an intestinal solution at pH=7.0.

Thus, the considered initial volume of solution was 200 ml and 10 grams of the supplemented food were introduced for each test at TO.

After 30 min, a 5 mL sample was taken and centrifuged at 4000 rpm for 40 min at T=4° C. The zinc contained in the supernatant (zinc ions and chelated zinc ions in solution) was measured by ICP-MS. The zinc contained in the remainder was also measured to verify general preservation.

Two zinc oxides and a zinc sulfate were compared to the ZnO—MgO premix in in vitro digestion. The percentage of solubilized zinc was measured after 30 minutes in order to compare the different zinc salts. Indeed, in order to overcome stomach complexation by phytates, the delay in stomach solubilization is defined as an efficiency factor in the context of the invention. A zinc salt is considered more effective if it is solubilized more slowly.

### Results

The results are presented in Table 1.

TABLE 1
Percentage of dissolution of the premix of the
invention and zinc oxide of the prior art

| | Total solubilized zinc |
|---|---|
| Source of Zinc | ions (120/30 min) %/% |
| Zinc sulfate | 1 |
| standard ZnO - prior art | 1 |
| ZnO HSS - prior art | 1 |
| Zinc Glycinate | 1.16 |
| Premix of the invention MgO—ZnO | 1.83 |
| prepared according to Example 1 | |

### Conclusion

The ZnO—MgO premix of the invention allows to significantly delay the solubilization of zinc, in comparison with conventional zinc oxides.

The synergistic premix comprising a zinc oxide and a magnesium oxide improves the penetration of zinc through intestinal cells in vitro, and demonstrates better availability compared to a Zn sulfate, a Zn glycinate and a ZnO. The synergy of action between zinc and magnesium allows to delay the kinetics of solubilization of zinc and to limit its complexation by the organic matter of the ration at the stage of digestion in the stomach, in particular phytates. The premix of the invention therefore allows to maximize the probability of absorption of zinc at the duodenum by the synergy between zinc and magnesium, which acts as a support and a means for physical protection of zinc in the digestive tract.

### Example 6: Absorption Test on Caco2 Cells of the Premix According to the Invention ZnO—MgO, and Comparison with the Prior Art

The objective of this test is to determine the absorption of minerals solubilized on a differentiated cellular permeable intestinal membrane (Caco2). The samples were obtained at the end of the in-vitro digestion DIV02 during which the ileal juices were collected and used on cells. The DIV allowed to evaluate the solubilization of zinc for the control (ZnSO4), for a zinc glycinate, for a zinc oxide with high specific surface (ZnO HSS) and for the ZnO—MgO premix.

Zinc sulfate, zinc glycinate and high specific surface zinc oxide of Hizox® brand were compared to the ZnO—MgO premix for this test in order to validate or invalidate the assumptions made during the development of the specifications to obtain product performances.

### Protocol

Immortal tumor cells are cultured in an incubator. Multiplication takes place under controlled atmosphere, temperature and light. Cells are placed on a membrane simulating the intestinal wall (0.4 μm).

The test was carried out on Caco2 cultured on insert and differentiated into representative intestinal monolayers after 17 days.

Ileal juices were deposited at the apical pole of the cells and after 60 minutes of incubation, the basal pole was removed entirely in order to measure the zinc concentration by ICP-MS.

### Results

The results are presented in Table 2.

TABLE 2
Zinc absorption on caco2 cells (mg/L) of the premix
of the invention and zinc sources of the prior art

| | Total Zn absorbed on cell |
|---|---|
| Source of Zinc | (mg/L) |
| Zinc sulfate | 0.67 |
| Zinc Glycinate | 0.28 |
| ZnO HSS - prior art | 0.74 |
| Premix of the invention MgO—ZnO | 1.89 |
| prepared according to Example 1 | |

The differentiated Caco2 cell test showed that zinc sulfate and high specific surface zinc oxide are both absorbed at 0.5-1 mg/L while the use of zinc glycinate only allows zinc absorption limited to 0.275 mg/L. On the other hand, the premix of the invention ZnO—MgO allows to reach 1.89 mg/L of total zinc absorbed on average. Three repetitions on this source were carried out.

### Example 7: In Vivo Evaluation of the Premix According to the Invention ZnO—MgO

The effect of the premix of the invention on the performance and health of piglets in the post-weaning pre-starter stage (0-14 days post-weaning) was evaluated and compared to a standard oxide.

The test is conducted in accordance with appropriate quality standards. The experimental procedures used in this test are approved by the RDN Animal Ethics Committee and are in accordance with Directive 2010/63/EU of the European Parliament and Council and with the Spanish recommendations for the care and use of animals for research (Boletin Official State, 2013).

### Doses and Products Tested:

This test was carried out with the premix according to the invention ZnO—MgO marketed under the brand CAPMAG®, at two levels of inclusion in the feed and compared to a standard ZnO at a dose of 3261 ppm and a standard ZnO at a dose of 150 ppm. All rations are formulated in accordance with the current regulations for piglets (FEDNA, 2013). The rations are distributed by RDN in the form of mash. The rations will be prepared by OCIPSA SIGLO XXI (Fuene de Cantos, Badajoz, Spain). All piglets receive a common commercial feed without innovative supplementation.

The use of the product of the invention ZnO—MgO was compared with standard zinc oxide at nutritional dose as well as at pharmacological dose in the contents indicated in Table 3.

TABLE 3
Concentrations used for each of the four
modalities tested during the test

| | Zinc content | Additive Content |
|---|---|---|
| Additive | (ppm) | (ppm) |
| 1 - ZnO positive control | 2620 | 3261 |
| 2 - ZnO negative control | 120 | 150 |
| 3 - ZnO—MgO (Low Dose) | 88 | 215 |
| 4 - ZnO—MgO (High Dose) | 133 | 325 |

### Animals:

A total of 288 piglets (Danbred×Duroc) weaned at 24±5 days of age are used for the test. Each piglet is tagged, weighed and allocated in groups of 8 piglets per pen. In total, 36 pens of dimensions 2.60×1.55 meters are considered for the test. The pens are distributed within three identical rooms with a controlled environment. All pens are equipped with an individual feeder and a drinking nipple. The piglets are distributed per pen so as to have a similar average weight between the different pens, an equal distribution of males and females and a personal space of 0.5 m² per piglet at 23 kg of weight (in accordance with RD 53/2013). Any preventive treatment with antibiotics or antimicrobials is avoided before the start of the test.

The experimental conditions of the experimental building are automatically controlled according to the age of the piglet and adapted to commercial practices.

Piglets are vaccinated against Mycoplasma and Circovirus.

### Measured Parameters: Weight Gain and Feed Consumption

Piglet weight was measured from D0 to D42 post-weaning in order to record daily weight gain and total weight at the end of the test. Feed consumption of each animal was also recorded each day of the test and recorded as an average to assess the feed conversion ratio (FCR). The FCR corresponds to the conversion index CI. It is equal to the average daily consumption reported to the average daily weight gain. The results are presented in Table 4. The mean values+/−the standard deviation are presented per treatment.

TABLE 4
Effects of nutritional treatments on the growth performance
of piglets from D 0 to D 42 post-weaning.

| Control | | ZnO—MgO | | SEM | |
|---|---|---|---|---|---|
| Positive | Negative | Low dose | High dose | N = 9 | P-value |

Live weight (kg)

| | | | | | | |
|---|---|---|---|---|---|---|
| Day 0 | 6.74 | 6.75 | 6.73 | 6.77 | 0.076 | 0.997 |
| Day 14 | 10.01^{ab} | 9.61^{b} | 10.31^{a} | 10.33^{a} | 0.169 | 0.022 |
| Day 42¹ | 25.33^{b} | 26.15^{ab} | 27.01^{ab} | 27.49^{a} | 0.518 | 0.030 |

Pre-starter phase (D 0 to D 14 post-weaning)

| | | | | | | |
|---|---|---|---|---|---|---|
| Average daily feed consumption (kg/d) | 0.395 | 0.380 | 0.412 | 0.409 | 0.010 | 0.180 |
| Average daily weight gain (kg/day) | 0.233^{ab} | 0.205^{b} | 0.256^{a} | 0.255^{a} | 0.008 | <.001 |
| Feed conversion ratio (kg/kg) | 1.705^{b} | 1.863^{a} | 1.617^{b} | 1.613^{b} | 0.034 | <.001 |

Starter Phase (D 15 to D 42 post-weaning)¹

| | | | | | | |
|---|---|---|---|---|---|---|
| Average daily weight gain (kg/day) | 0.571^{b} | 0.611^{ab} | 0.621^{a} | 0.618^{a} | 0.011 | 0.013 |

Overall period (D 0 to D 42 post-weaning)¹

| | | | | | | |
|---|---|---|---|---|---|---|
| Daily weight gain (kg/day) | 0.444^{b} | 0.463^{ab} | 0.486^{a} | 0.482^{a} | 0.009 | 0.011 |

¹Live weights of piglets euthanized at D 14 of the test for ileal tissue analyses were removed from the dataset used for growth performance results analyzed from D 15 to D 42 post-weaning.

### Analysis of the Results:

Piglets receiving the product ZnO—MgO had higher live weights than piglets receiving standard ZnO at nutritional dose, but also higher than those receiving standard ZnO at pharmacological dose.

The product of the invention ZnO—MgO significantly improved the average daily weight gain. The supply of ZnO—MgO did not show any impact on the feed consumption of piglets. The use of ZnO—MgO premix as a source of zinc in the premix significantly improved the consumption index compared to the negative control.

Piglets consuming ZnO—MgO continue to gain more weight daily in the starter phase following the pre-starter phase of supplementation. Thus, the use of ZnO—MgO as supplementation on the pre-starter phase has a prolonged impact on the animal.

### Feces Analysis:

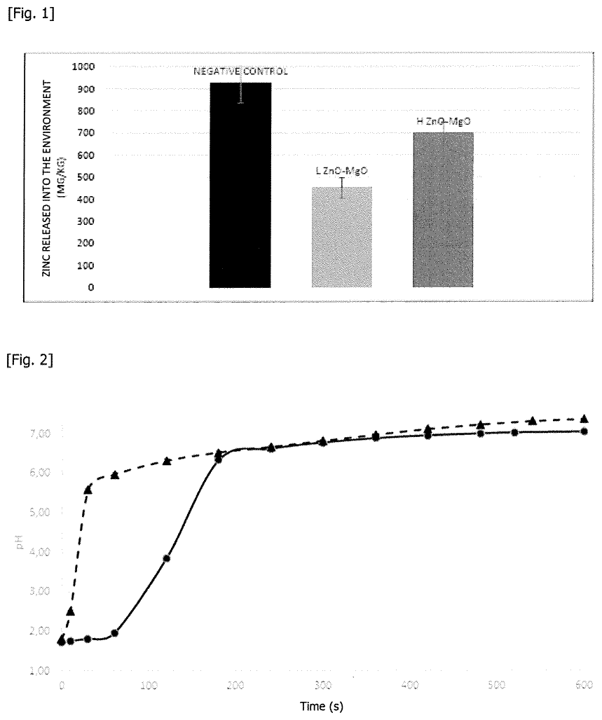

Analyses of the faeces after 14 days of use of the different treatments allowed to assess the amount of zinc not used by the animal and thus released into the soil, contributing to pollution. The results are presented in FIG. 1.

The use of the ZnO—MgO mixture in post-weaning piglets during this in-vivo test allowed a reduction in the release of the element zinc into the soil. The content measured in the feces is respectively 450 ppm and 697 ppm for the low dose (L ZnO—MgO) and the high dose (H ZnO—MgO) of the premix. These amounts of zinc are lower than that of 925 ppm released by piglets having been supplemented with standard ZnO.

### Example 8: Dissolution Kinetics of the Product of the Invention and of a Comparative Product, in an Acid Medium

The kinetics of pH evolution as a function of time of the product according to Example 1 was compared with that of a comparative product.

### Products Evaluated:

The product of Example 1 was obtained by heating at a temperature of 1000° C. a powdery mixture of a magnesium oxide raw material and a trace element oxide raw material. The comparative product was obtained by mixing at room temperature a powdery raw material of magnesium oxide and a powdery trace element oxide raw material. The comparative product was identical to the raw material mixture that was used to prepare the product of Example 1. The comparative product therefore did not undergo any heat treatment at a temperature higher than 700° C.

### Dissolution Protocol:

The dissolution protocol is identical to that described above.

### Results:

The results are shown in FIG. 2, the dotted curve corresponding to a mixture not in accordance with the invention.

The evolution of pH is directly correlated to the solubilization of the crystalline phases present in the hydrochloric medium. Thus, the kinetics of pH evolution allows to follow the kinetics of release of Zn²⁺ (and therefore Mg²⁺) in the reaction medium.

The non-solubilization of the premix of the invention (solid curve) between 0 and 60 seconds is followed by a delay extending up to 200 seconds of reaction, which allows to consider a bypass with respect to the antagonists in the digestive tract.

On the other hand, it is observed that a comparative product, which is not in the form of a ternary solid—comprising a zinc oxide and a magnesium oxide and having two different crystalline forms (dotted curve)—does not cause any delay in dissolution and undergoes very rapid, almost instantaneous solubilization in an acid medium.

1. A premix for animal nutrition comprising a ternary solid of magnesium, at least one trace element and oxygen, said trace element being selected in the group consisting of iron, copper, zinc and manganese.

2. The premix according to claim 1, wherein magnesium and oxygen forms magnesium oxide, the ternary solid being a substitution solid solution of the trace element in magnesium oxide.

3. The premix according to claim 1, wherein the trace element represents from 10% to 45% by mole of the ternary solid.

4. A process for manufacturing a premix for animal nutrition, said process comprising a step of heating, at a temperature ranging from 700° C. to 1500° C., a mixture comprising at least one powdery raw material of a magnesium oxide, and at least one powdery raw material of a trace element.

5. The process for manufacturing according to claim 4, wherein a particle size of the at least one powdery raw material of a magnesium oxide and the particle size of at least one powdery raw material of a trace element are less than 200 microns.

6. The process for manufacturing according to claim 4, wherein the purity of the powdery raw material of a magnesium oxide and the purity of the powdery raw material of a trace element are greater than 95% by mass.

7. The process for manufacturing according to claim 4, wherein the powdery raw material of a magnesium oxide is selected in the group consisting of a magnesia, a magnesium carbonate, a magnesium hydroxycarbonate, a magnesium sulfate, a magnesium phosphate, a magnesium chloride, a magnesium hydroxide and one of their hydrates.

8. The process for manufacturing according to claim 4, wherein the powdery raw material of a trace element raw material is a trace element oxide raw material comprising a compound selected from in the group consisting of ZnO, ZnCO₃, Zn₅(CO₃)₂(OH)₆, ZnSO₄, FeCO₃, FeO, Fe₂O₃, Fe₃O₄, FeHO₂, Fe₄H₆Og, MnCO₃, MnO, Mn₂O₃, MnO₂, Mn₃O₄, Mn(OH)₂, CuCO₃, CuO, Cu₂O and Cu(OH)₂.

9. The process for manufacturing according to claim 7, wherein the powdery raw material of a magnesium oxide is a magnesia having a particle size value D50 that is less than 100 microns.

10. (canceled)

11. A method for producing at least one biological effect selected in the group consisting of increasing the weight gain of the animal, supporting the growth of the animal, improving feed efficiency, improving the feed conversion index and supporting the assimilation of nutrients, in a non-human animal, said method comprising a step of feeding the animal.

12. The method according to claim 11, wherein the non-human animal is selected in the group consisting of livestock, racing animals, and domestic animals.

13. A nutritional supplement for non-human animals comprising the premix according to claim 1, and a compound selected from vitamins, probiotics, macroelement salts, trace element salts, enzymes and amino acids.

14. A process for preparing a feed ration for a non-human animal, comprising a step of dry incorporation of the premix according to claim 1, into fodder or cereals.
